(19) United States
(12) Patent Application Publication
(10) Pub. No.: US 2024/0369549 A1
(43) Pub. Date: Nov. 7, 2024

(54) APTAMER-BASED ELECTROCHEMICAL VIRUS DETECTOR AND METHODS THEREOF

(71) Applicant: THE JOHNS HOPKINS UNIVERSITY, Baltimore, MD (US)

(72) Inventors: Yuxuan WANG, Baltimore, MD (US); Chao WANG, Ellicott City, MD (US); Hongpeng JIA, Baltimore, MD (US); Mukund Madhav GOYAL, Silver Spring, MD (US)

(21) Appl. No.: 18/689,342

(22) PCT Filed: Jul. 27, 2022
(86) PCT No.: PCT/US2022/038491

§ 371 Date: Mar. 5, 2024

Related U.S. Application Data
(60) Provisional application No. 63/243,889, filed on Sep. 14, 2021.

(51) Int. Cl. C12Q 1/06; G01N 27/327; G01N 33/543; G01N 33/569

(52) U.S. Cl. CPC C12Q 1/06; G01N 27/3276; G01N 33/5438; G01N 33/56983; G01N 2333/165

(57) ABSTRACT

A method for virus detection is disclosed, including introducing a biological media to an aptamer functionalized electrochemical sensor, generating an electrochemical signal from a reaction between a virus and an aptamer, analyzing the electrochemical signal to determine presence of the virus, analyzing the electrochemical signal to determine a quantity of the virus, and transmitting a presence of the virus and a quantity of the virus. A device for virus detection and method for fabricating a device for virus detection is also disclosed. The device for virus detection also includes an electrode, a functionalized aptamer anchored to the electrode, where an aptamer is functionalized such that it is specific to a virus, such as SARS-COV-2 or an alternate virus or interest. The device for virus detection may be wearable.

### CROSS-REFERENCE TO RELATED APPLICATIONS

This application is the national stage entry of International Patent Application No. PCT/US2022/038491, filed on Jul. 27, 2022, and published as WO 2023/043540 A1 on Mar. 23, 2023, which claims the benefit of U.S. Provisional Patent Application Ser. No. 63/243,889, filed Sep. 14, 2021, the disclosures of which are incorporated herein by reference in their entireties.

### TECHNICAL FIELD

The present teachings relate generally to a virus detector and, more particularly, to aptamer-based electrochemical virus detectors, associated methods, and devices.

### BACKGROUND

Rapid or fast-pre-screening techniques for infectious disease are essential in informing and containing pathogenic outbreaks or pandemics. In times of disease outbreaks, if a person found themselves positive for a pathogenic or viral disease with an at-home device, they could self-quarantine, travel to a nearby facility for more precise testing, or convey test results to a medical professional. Without such a rapid screening technique, a patient would have to wait overnight and sometimes days before receiving their test results. By the time a positive result is received, the patient may have potentially already infected many others at home or in public places, such as grocery stores, subway stations, schools, and the like.

If this a small-scale, rapid pre-screening device were available, outbreaks such as the COVID-19 outbreak may have been more containable and informed. In other environments, such as in the developing world, a small scale, cheap virus detecting device is also important, due to a lack of available laboratory resources. Such devices could potentially save thousands of lives.

Thus, a method of and apparatus for detecting and/or sensing viruses that is small scale, inexpensive, and reagent-less, while providing rapid results to a person or medical professional would be desirable.

### SUMMARY

The following presents a simplified summary in order to provide a basic understanding of some aspects of one or more embodiments of the present teachings. This summary is not an extensive overview, nor is it intended to identify key or critical elements of the present teachings, nor to delineate the scope of the disclosure. Rather, its primary purpose is merely to present one or more concepts in simplified form as a prelude to the detailed description presented later.

A method for virus detection is disclosed. The method for virus detection also includes introducing a biological media to an aptamer functionalized electrochemical sensor. The method for virus detection also includes generating an electrochemical signal from a reaction between a virus and an aptamer. The method for virus detection also includes analyzing the electrochemical signal to determine presence of the virus. The method for virus detection also includes analyzing the electrochemical signal to determine a quantity of the virus. The method for virus detection also includes transmitting a presence of the virus and a quantity of the virus.

The method for virus detection may include implementations where the aptamer is functionalized such that it is specific to a virus. The aptamer may be functionalized with methylthioninium chloride. The biological media may be an aerosol. The biological media may be a liquid. The biological media may be blood. The biological media may be saliva. The method for virus detection may include calibrating an electrochemical signal to a presence of a virus included in the biological media in units of virus particles per milliliter. The aptamer functionalized electrochemical sensor has a limit of detection of 10,000 virus particles per milliliter. The method for virus detection may include receiving a presence of the virus and a quantity of the virus via a display, secondary device, or combination thereof.

A method for fabricating a device for virus detection is disclosed. The method for fabricating a device for virus detection also includes functionalizing a portion of an electrode surface with a virus-specific aptamer. The method for fabricating a device for virus detection also includes passivating a remainder of the electrode surface with a binder molecule, and where the virus-specific aptamer is functionalized on a first end with a redox couple, and the virus-specific aptamer is functionalized on a second end with a surface-reactive group.

Implementations for fabricating a device for virus detection may include one or more of the following features. The method for fabricating a device for virus detection may include where the electrode surface may include gold. The redox couple in the device for virus detection responds to methylthioninium chloride. The binder molecule may include mercapto-1-hexanol. The surface-reactive group may include a mercapto group.

A device for virus detection is disclosed. The device for virus detection also includes an electrode. The device for virus detection also includes a functionalized aptamer anchored to the electrode, where an aptamer is functionalized on a first end with a redox couple and the aptamer is functionalized on a second end with a surface-reactive group. The device for virus detection also includes a binder molecule anchored to the electrode.

Implementations of the device for virus detection may include one or more of the following features. The device for virus detection may include a wearable article upon which the electrode is disposed. The functionalized aptamer may be functionalized such that it is specific to a virus. The virus may be SARS-COV-2. The redox couple may be methylthioninium chloride. The binder molecule may include mercapto-1-hexanol. The device for virus detection may include a display coupled to the electrode.

The features, functions, and advantages that have been discussed can be achieved independently in various implementations or can be combined in yet other implementations further details of which can be seen with reference to the following description.

### BRIEF DESCRIPTION OF THE DRAWINGS

The accompanying drawings, which are incorporated in and constitute a part of this specification, illustrate embodiments of the present teachings and together with the description, serve to explain the principles of the disclosure. In the figures:

FIG. 1 depicts a schematic overview illustrating the fabrication and function of an aptamer-based electrochemical virus sensor, according to and embodiment.

FIGS. 2A and 2B are photographs under magnification illustrating a microstructure of a substrate coated with gold via an electrodeposition method and a sputtering method, respectively.

FIG. 3 is a plot of an electrochemical signal generated utilizing an aptamer-based electrochemical virus sensor, according to an embodiment.

FIG. 4 is a plot of a calibration curve of an electrochemical signal generated utilizing an aptamer-based electrochemical virus sensor, according to an embodiment.

FIG. 5 is a plot illustrating a virus specificity test showing signal response using an aptamer-based electrochemical virus sensor, according to and embodiment.

FIG. 6 is a flowchart illustrating a method of detecting a virus with an aptamer-based electrochemical virus sensor, according to an embodiment.

It should be noted that some details of the figures have been simplified and are drawn to facilitate understanding of the present teachings rather than to maintain strict structural accuracy, detail, and scale.

### DETAILED DESCRIPTION

Reference will now be made in detail to exemplary embodiments of the present teachings, examples of which are illustrated in the accompanying drawings. Wherever possible, the same reference numbers will be used throughout the drawings to refer to the same, similar, or like parts.

Embodiments described herein include a method of and apparatus for detecting, sensing or reporting viruses that maintains a small scale, is inexpensive and reagent-less, and provides rapid results to an individual or medical professional. The electrochemical virus sensor as described herein is small in scale, cheap, reagent-less and fast. With these design attributes, an economically friendly, portable virus testing device can be realized. While there are commercially available electrochemical systems for virus detection, the devices described herein may be wireless, and sized on the order of a common flash drive. The cost of building may be quite low as well. Virus sensors as described may be integrated with small, cheap electrochemical sensors, or other accompanying devices, to address the problem of virus pre-screening.

Embodiments of virus detectors and associated methods described herein utilize aptamers. An aptamer is a stable DNA or RNA ligand that is designed to bind strongly and with high affinity to specific target molecules, for example, proteins, peptides, carbohydrates, small molecules, toxins, live cells, and virus proteins. The aptamer strand for the device disclosed herein has been modified to have a thiol functional group on one end of the aptamer and a redox reporter, also referred to as a redox indicator at the other end. In one embodiment as described herein, the redox reporter may be, but is not limited to methylene blue, or methylthioninium chloride. The aptamer strand is selected to strongly bond to a specific virus protein. In one specific embodiment, as described herein, the aptamer has been designed to bind with a SARS-CoV-2 spike protein pseudotyped FIV, although in other embodiments other viral proteins or components may be the aptamer target. The thiol group allows the aptamer to be functionalized on an electrode, such as gold, and the redox couple may be utilized as a reporter to provide an electrochemical signal. Once the specific, targeted virus is chemically bonded to the aptamer strand, the distance of the methylene blue to the electrode surface will be changed, thus affecting the electron transfer property of the methylene blue, or methylthioninium chloride redox reaction. This virus sensing can thus be integrated onto a small electrochemistry module to indicate a presence or absence of the targeted virus. Successful demonstration of two applications of this electrochemical virus sensing technique has been conducted, from virus containing analyte dropcast and from aerosol. These two applications may enable use of the virus detection method and device described herein towards a small scale, portable diagnostic device, that may be integrated into an at-home test kit and everyday consumer product, such as masks or other wearable elements. The detection limit of preliminary studies using the virus detector having an aptamer designed to specifically bind with a SARS-COV-2 (COVID-19) spike protein pseudotyped FIV is below 1000 VP/ml, which is far below a typical virus level found in a saliva sample from a patient.

Typical disadvantages of electrochemical sensor are a compromised or inadequate LOD. As compared to the PCR technology, which is considered a gold standard in SARS-Cov-2 testing and having a detection limit as low as 1 VP/ml, typical electrochemical corona virus sensors can only detect 1 e⁸ VP/ml, far above the clinical level of 1 e⁶ VP/ml. This high LOD limits the commercialization of known electrochemical virus detector. The aptamer-based electrochemical sensors as described herein demonstrates an improved LOD, achieving virus detection with concentration as low as 1 e⁴ VP/ml. Without being bound by any particular theory, it is believed that this improvement is gained by several features. First, the aptamer molecules on the electrode surface are closely packed on the electrode surface, on the order of ˜1 e4 aptamers/square microns. Since there are multiple binding sites on a single virus, as well as multiple spike proteins on a virus particle, the binding strength between the target virus and the aptamer becomes much higher. This multiple binding mechanism fundamentally alters the thermodynamics of the aptamer-virus binding, which enables virus detection at higher sensitivity, well within the clinical range.

The second aspect of the aptamer-based electrochemical virus sensor as described herein is the application of an aerosol biosensor, by utilization of advanced nanotechnology. In order to detect virus from the aerosol, different nano-structuring techniques, such as gold sputtering on porous substrate, gold electrodeposition, and gold electrochemical roughening, as well as other methods. It has been demonstrated that by creating a porous nano-structure electrode, the sensor may physically capture the miniature virus particles more efficiently than as compared to a smooth gold electrode surface.

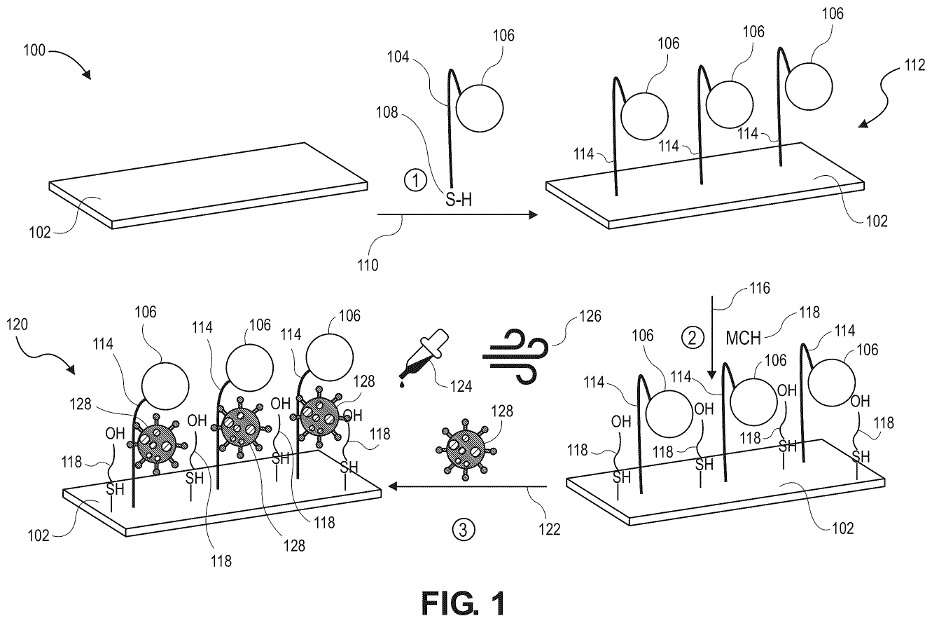

FIG. 1 depicts a schematic overview illustrating the fabrication and function of an aptamer-based electrochemical virus sensor, according to and embodiment. The fabrication of an aptamer-based electrochemical sensor 100 is shown, beginning with a substrate 102 which is a gold substrate for an electrode. Alternate embodiments may utilize other suitable metals or other materials known in the art for electrode substrates or substrates for electrochemical virus sensors as shown herein. Other suitable metals or other materials may include, but are not limited to, carbon nanotubes, graphene, Ordered mesoporous carbon, titanium, platinum, silver. In a substrate surface aptamer functionalization step 110, an aptamer 104 having an affinity for a specific target viral protein and functionalized on a first end with a methylene blue redox couple 106 and functionalized with a thiol or mercapto functional group 108 mercapto on a second, opposite end of the aptamer is exposed to the substrate. Alternate redox couples or redox reporters useful in embodiments may include ferrocene, inorganic functional groups, organic functional groups, or combinations thereof. Alternate anchoring functional groups as alternatives to the thiol or mercapto group useful in embodiments may include amine, isocyano, methylsulfide, isothiocyanate, selenol, pyridine, phosphine, pyrene, diazonium, organotin, trimethylsilyl, carboxyl, nitro, dithiacarboxyl, or combinations thereof. In certain embodiments, the aptamer may be spin coated onto a surface of the substrate. Aptamers for specific viruses or specific variants thereof may be purchased or developed specifically for use within alternate embodiments of the aptamer-based electrochemical sensor 100 as shown in FIG. 1. Other embodiments may be coated with the aptamer by means or procedures other than chemical bonding known to those skilled in the art, such as physical adsorption. But I'm skeptical about the binding strength. Alternatively, when a different electrode surface material is used, a corresponding functional group on the second end of the aptamer 104 may be used. The functionalizing of a portion of an electrode surface with a virus-specific aptamer results in an aptamer functionalized sensor 112 having a functionalized anchored aptamer 114 coated upon the substrate 102 surface. Next, in a surface functionalization step 116, the remainder of the electrode surface is passivated with a binder molecule, for example, mercapto-1-hexanol (MCH) 118 in order to reduce the unspecific binding to the electrode. This reaction of adding the electrochemical blocker mercapto-1-hexanol (MCH) 118 completes the surface assembled sensor 120. Next, a potential virus incubation step 122 may be conducted by an introduction of virus via drop-cast 124 or an introduction of virus via aerosol 126. The virus 128 may also be introduced in the absence of any biological media. Other means of introduction of biological samples possibly having a potential virus present in the biological sample may be used in alternative embodiments, such as, but not limited to, sneeze, saliva, blood, perspiration, vomit, sweat, urine, stool or combinations thereof. As the aptamer-based electrochemical sensor 100 is exposed to the virus 128, the concentration of the virus 128 can be measured electrochemically, as will be described later. The aptamer used in the devices and analyses described herein was manufactured by BasePair (https://www.basepairbio.com/covid19/) to specifically target COVID-19, also referred to as SARS-COV-2, however, the mercapto (thiol) group and the redox couple (methylene blue) were specifically customized as requested. Aerosol testing as described previously was utilized to test the virus in atmosphere, which provides demonstration for applications such as an electrochemical virus sensor on a wearable article, such as a mask, or an environmental virus sensor. Drop-cast testing was also successfully utilized as an analogue for saliva or other biological fluid testing. Additional elements to the aptamer-based electrochemical sensor 100 not shown herein may include the integration of such a virus detector into other wearable articles, such as, but not limited to, wrist straps, laboratory coats, interior or exterior cloth items, an entryway style device, a breathalyzer, test strips, handset, watch, bracelet, tooth braces, or external devices, displays, or other device coupled to the electrode, for a means of providing a visual, audible, or otherwise detectable signal to notify one of a presence or absence of the targeted virus. The aptamer-based electrochemical sensor 100 may be battery powered or powered by an external or alternate power source in certain embodiments.

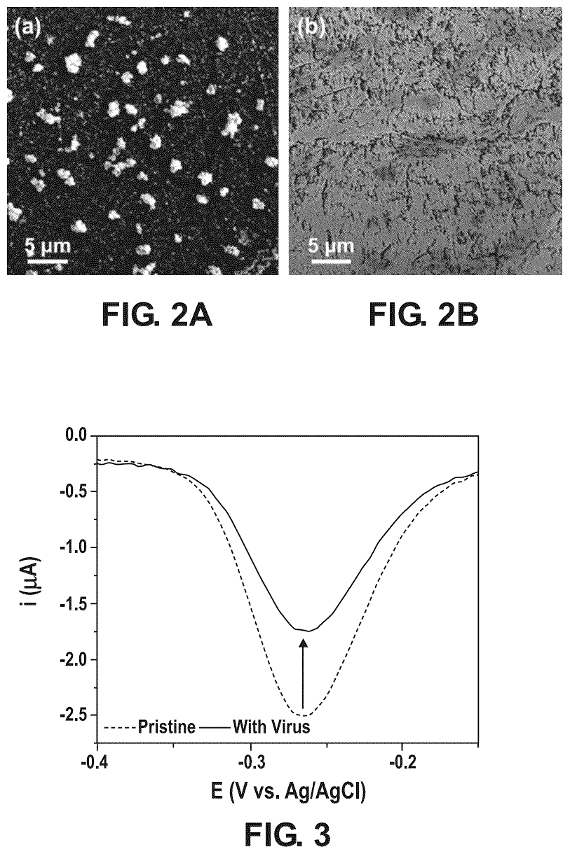

FIGS. 2A and 2B are photographs under magnification illustrating a microstructure of a substrate coated with gold via an electrodeposition method and a sputtering method, respectively. Substrates coated with gold to produce an electrode for aptamer-based virus detectors as described herein may have a surface nanostructure created to enable more successful capturing of a targeted virus in an aerosol or other sample to effectively conduct electricity. FIG. 2A exhibits gold nanoparticles deposited onto a substrate via a pulse-electrodeposition method. FIG. 2B shows a microporous electrode surface sputtered with gold. In this depicted embodiment, the substrate is the electrode material. The intended purpose of the nanoparticle deposition is to increase the density and/or surface area of the anchoring sites. Other methods for coating a substrate may include metal sputtering or evaporation, nanoparticle physical deposition, dealloying, annealing, and the like. It should be noted that nanostructures as shown may increase the binding sites that electrode can offer, which will may also increase the aptamer density on the electrode surface and thus improve sensitivity.

FIG. 3 is a plot of an electrochemical signal generated utilizing an aptamer-based electrochemical virus sensor, according to an embodiment. The plot shown in FIG. 3 was generated using square wave voltammetry in 1X PBS (pristine). The phosphate-buffer saline (PBS) is used to generate a control signal, as compared to a signal of an electrochemical signal obtained by exposing the aptamer-based virus detector to a concentration of 1000 virus particle/ml. The virus used in this demonstration is SARS-COV-2 spike protein pseudotyped Feline immunodeficiency virus (FIV). In the 1X PBS sample, the aptamer on the virus detector or sensor was not bonded to any analyte, and the peak current for the methylene blue redox reaction is at −2.5 μA. Once the aptamer is bonded to the virus as shown in the “with virus” current curve, the distance between the methylene blue and the electrode surface becomes greater, which results in a lower peak current of the methylene blue redox reaction. In this case, once the aptamer was bonded to the virus, the current decreases from −2.5 μA to −1.5 μA. The virus concentration delivered to the sensor can then be quantified by this change in peak current during a calibration procedure.

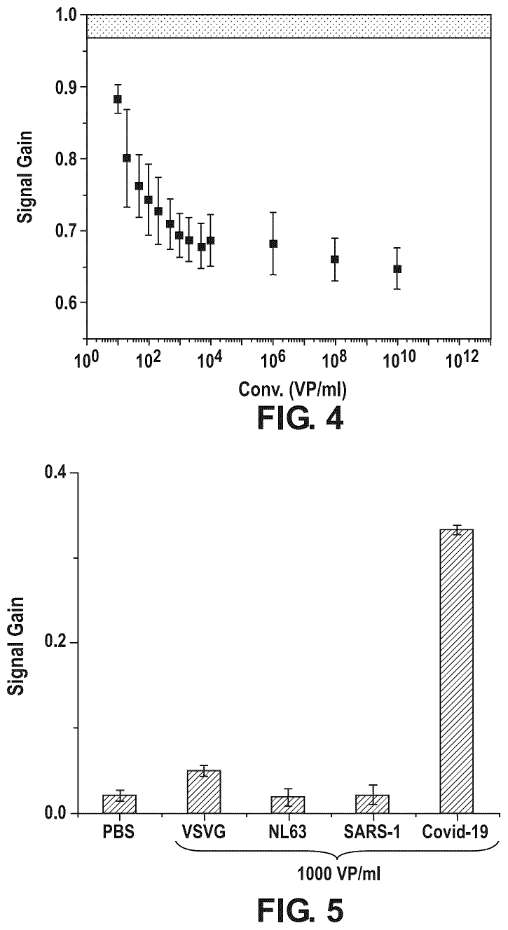

FIG. 4 is a plot of a calibration curve of an electrochemical signal generated utilizing an aptamer-based electrochemical virus sensor, according to an embodiment. By varying the known concentration of virus exposed to the aptamer-based electrochemical virus sensor similar to the data shown in FIG. 3, a calibration curve was generated with various levels of VP/ml. The aptamer-based electrochemical virus sensor showed very good signal as low as 10 VP/ml. The signal gain was calculated by the following calculation:

$$Signal gain = ( peak current in 1 X PBS and various virus concentration ) / ( peak current in 1 X PBS ) \qquad ( Eq . 1 )$$

The error bar was calculated from a standard deviation of three independent measurements. The upper shaded area shows the limit of detection (LOD) of the method as measured, beyond which the concentration range will exhibit a p-value of <0.05. The LOD was calculated as follows:

$$LOD = meanblank + 1.645 ( SDblank ) + 1.645 ( SDlowconcentration ) \qquad ( Eq . 2 )$$

Where SDblank is the standard deviation at 1X PBS and SD low concentration is the standard deviation measured at low virus concentration. In general, the aptamer-based electrochemical virus sensor exhibits a demonstrated response from 10 VP/ml and an effective dynamic range of detection from 10 VP/ml to 106 VP/ml. The demonstrated signal gain, exhibited as a reduction from about 1.0, or zero detection, to about as low as 10 VP/ml and within a high sensitivity, or preclinical detection range. These levels are much lower than other known methods, providing measurement results within a few minutes or less. The error bars illustrated in the plot of FIG. 4 may demonstrate that the method has a reliability of 10% or less.

FIG. 5 is a plot illustrating a virus specificity test showing signal response using an aptamer-based electrochemical virus sensor, according to and embodiment. As the aptamer used in the aptamer-based electrochemical virus sensor is deigned to specifically target COVID-19, the aptamer-based electrochemical virus sensor only shows appreciable signal response with COVID-19 pseudo-type virus samples. The alternate sample are as follows-VSVG is a pseudo virus with Vesicular stomatitis virus G (VSV G) protein, NL63, SARS-lare pseudotyped FIV contains spike protein from human corona virus NL63 or SARS-COV-1. As a suitable frame of reference, the range of 10 e6-10 e7 is a typical range of viral concentration or viral load for COVID-19 where symptoms would begin to appear in a patient. Again, the data illustrated in FIG. 5 shows the selectivity of this diagnostic method to the COVID-19 spike protein, reducing false positives as well. The aptamer used in this particular design of electrochemical sensor is specific to the SARS-COV2 coronavirus.

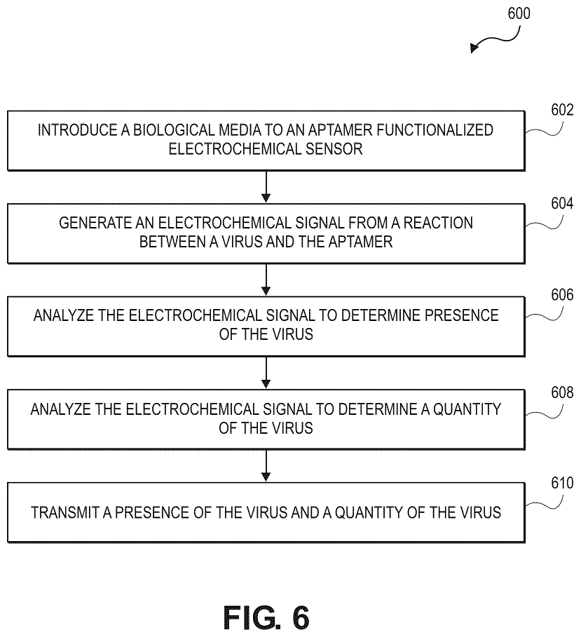

FIG. 6 is a flowchart illustrating a method of detecting a virus with an aptamer-based electrochemical virus sensor, according to an embodiment. As shown in FIG. 6, a method for virus detection 600 is shown, with a first step being to introduce a biological media to an aptamer functionalized electrochemical sensor 602 which in turn will generate an electrochemical signal from a reaction between a virus and the aptamer 604. The next step is to analyze the electrochemical signal to determine presence of the virus 606, followed by a step to analyze the electrochemical signal to determine a quantity of the virus 608. The information obtained, after analysis, may be transmitted, in terms of a presence of the virus and a quantity of the virus 610.

In the method for virus detection 600, the aptamer is functionalized such that it is specific to a virus. The aptamer may be functionalized to specifically target SARS-COV-2 or an analogue thereof. In certain embodiments, a different aptamer design may be used to target other viruses such as, but not limited to, human or other corona viruses, repiratory syncytial virus (RSV), rhinoviruses, influenza viruses, human immunodeficiency virus (HIV) and other retroviruses, ebola virus, Zeka virus, and other emerging or re-emerging viruses of various transmittables and variants. The aptamer in the method for virus detection 600 may be functionalized with methylthioninium chloride as a redox reporter or redox indicator. The aptamer in the method for virus detection 600 may be functionalized with a thiol functional group. In the method for virus detection 600, the biological media introduced to the sensor may be in aerosol form, liquid form, or combinations thereof. In the method for virus detection 600, the biological media introduced to the sensor may be phlegm, mucous, sneeze, blood, urine, sweat, vomit, saliva, aspirated saliva, stool, and other biopsy samples, or combinations thereof. The method for virus detection 600 may also include calibrating an electrochemical signal to a presence of a virus included in the biological media in units of virus particles per milliliter, as described previously herein. In certain embodiments, the aptamer functionalized electrochemical sensor used in the method for virus detection 600 has a limit of detection of 10,000 virus particles per milliliter. The method for virus detection 600 may include receiving a presence of the virus and a quantity of the virus via a display, secondary device, or a combination thereof. The method for virus detection 600 may also include presenting a virus detection device or virus sensor as described herein to a patient, exposing a biological sample from the patient to the detection device, and generating an electrochemical signal using a redox reporter, such as methylene blue, if the target virus is present, wherein the virus bonds to the virus sensor surface indicating a positive test result.

While the present teachings have been illustrated with respect to one or more implementations, alterations and/or modifications may be made to the illustrated examples without departing from the spirit and scope of the appended claims. For example, it may be appreciated that while the process is described as a series of acts or events, the present teachings are not limited by the ordering of such acts or events. Some acts may occur in different orders and/or concurrently with other acts or events apart from those described herein. Also, not all process stages may be required to implement a methodology in accordance with one or more aspects or embodiments of the present teachings. It may be appreciated that structural objects and/or processing stages may be added, or existing structural objects and/or processing stages may be removed or modified. Further, one or more of the acts depicted herein may be carried out in one or more separate acts and/or phases. Furthermore, to the extent that the terms “including,” “includes,” “having,” “has,” “with,” or variants thereof are used in either the detailed description and the claims, such terms are intended to be inclusive in a manner similar to the term “comprising.” The term “at least one of” is used to mean one or more of the listed items may be selected. Further, in the discussion and claims herein, the term “on” used with respect to two materials, one “on” the other, means at least some contact between the materials, while “over” means the materials are in proximity, but possibly with one or more additional intervening materials such that contact is possible but not required. Neither “on” nor “over” implies any directionality as used herein. The term “conformal” describes a coating material in which angles of the underlying material are preserved by the conformal material. The term “about” indicates that the value listed may be somewhat altered, as long as the alteration does not result in nonconformance of the process or structure to the illustrated embodiment. The terms “couple,” “coupled,” “connect,” “connection,” “connected,” “in connection with,” and “connecting” refer to “in direct connection with” or “in connection with via one or more intermediate elements or members.” Finally, the terms “exemplary” or “illustrative” indicate the description is used as an example, rather than implying that it is an ideal. Other embodiments of the present teachings may be apparent to those skilled in the art from consideration of the specification and practice of the disclosure herein. It is intended that the specification and examples be considered as exemplary only, with a true scope and spirit of the present teachings being indicated by the following claims.

1. A method for virus detection, comprising:
introducing a biological media to an aptamer functionalized electrochemical sensor;
generating an electrochemical signal from a reaction between a virus and an aptamer;
analyzing the electrochemical signal to determine presence of the virus;
analyzing the electrochemical signal to determine a quantity of the virus; and
transmitting a presence of the virus and a quantity of the virus.

2. The method for virus detection of claim 1, wherein the aptamer is functionalized such that it is specific to a virus.

3. The method for virus detection of claim 1, wherein the aptamer is functionalized with methylthioninium chloride.

4. The method for virus detection of claim 1, wherein the biological media is an aerosol.

5. The method for virus detection of claim 1, wherein the biological media is a liquid.

6. The method for virus detection of claim 1, wherein the biological media is blood.

7. The method for virus detection of claim 1, wherein the biological media is saliva.

8. The method for virus detection of claim 1, further comprising calibrating an electrochemical signal to a presence of a virus included in the biological media in units of virus particles per milliliter.

9. The method for virus detection of claim 1, Wherein the aptamer functionalized electrochemical sensor has a limit of detection of 10,000 virus particles per milliliter.

10. The method for virus detection of claim 1, further comprising receiving a presence of the virus and a quantity of the virus via a display, secondary device, or combination thereof.

11. A method for fabricating a device for virus detection, comprising:
functionalizing a portion of an electrode surface with a virus-specific aptamer;
passivating a remainder of the electrode surface with a binder molecule; and wherein:
the virus-specific aptamer is functionalized on a first end with a redox couple; and
the virus-specific aptamer is functionalized on a second end with a surface-reactive group.

12. The method for fabricating a device for virus detection of claim 11, wherein the electrode surface comprises gold.

13. The method for fabricating a device for virus detection of claim 11, wherein the redox couple responds to methylthioninium chloride.

14. The method for fabricating a device for virus detection of claim 11, wherein the binder molecule comprises mercapto-1-hexanol.

15. The method for fabricating a device for virus detection of claim 11, wherein the surface-reactive group comprises a mercapto group.

16. A device for virus detection, comprising:
an electrode;
a functionalized aptamer anchored to the electrode; wherein an aptamer is functionalized on a first end with a redox couple and the aptamer is functionalized on a second end with a surface-reactive group; and
a binder molecule anchored to the electrode.

17. The device for virus detection of claim 16, further comprising a wearable article upon which the electrode is disposed and the electrode is coupled to the electrode.

18. The device for virus detection of claim 16, wherein the functionalized aptamer is functionalized such that it is specific to a virus.

19. The device for virus detection of claim 18, wherein the virus is SARS-COV-2.

20. The device for virus detection of claim 16, wherein:
the redox couple is methylthioninium chloride; and
the binder molecule comprises mercapto-1-hexanol.

21-22. (canceled)
